(19) United States
(12) Patent Application Publication
(10) Pub. No.: US 2023/0215541 A1
(43) Pub. Date: Jul. 6, 2023

(54) SMART SCHEDULING AND INFORMATION GATING SYSTEMS AND METHODS TO PROMOTE SLEEP AND MENTAL HEALTH

(71) Applicant: KONINKLIJKE PHILIPS N.V., Eindhoven (NL)

(72) Inventors: Boomika KALYAN, Pittsburgh, PA (US); Gary Nelson GARCIA MOLINA, Madison, WI (US); Sara KRON, Pittsburgh, PA (US)

(21) Appl. No.: 17/565,819

(22) Filed: Dec. 30, 2021

(51) Int. Cl. G16H 20/70

(52) U.S. Cl. CPC G16H 20/70

(57) ABSTRACT

A smart scheduling and information gathering system to promote sleep and mental health in unprecedented emergency situations. The system includes a remote server used for gathering contextual event data from external data sources, capturing physiological data from an environmental sensor, generating a sleep availability rating by analyzing a sleep activity log, generating a scheduling availability rating by analyzing a current-date-and-time in relation to a user calendar managed by the remote server, and generating a behavioral recommendation and a filtering protocol by analyzing the contextual event data, the physiological data, the sleep availability rating, and the scheduling availability rating. The system further includes a user computing device used for outputting the behavioral recommendation, comparing incoming data to the filtering protocol in order to classify the incoming data as authorized data or unauthorized data, and outputting the authorized data.

### CROSS-REFERENCE TO PRIOR APPLICATIONS

This application claims the benefit of U.S. application Ser. No. 17/218,234, filed on 31 Mar. 2021. This application is hereby incorporated by reference herein.

### BACKGROUND OF THE INVENTION

### 1. Field of the Invention

The present invention generally relates to systems and methods used to suggest lifestyle changes for improved mental health.

### 2. Description of the Related Art

On 30 Jan. 2020, the World Health Organization (WHO) declared the novel coronavirus outbreak an international emergency, and on Mar. 11, 2020, the WHO declared the global COVID-19, the disease caused by the virus, a pandemic. As of Apr. 23, 2020, almost 2.7 million COVID-19 cases had been confirmed globally with over 185,000 confirmed global deaths. As these numbers continue to increase, governments of countries across the world are rapidly implementing measures to minimize the risk of exposure and spread in efforts to flatten the curve of community transmission.

As part of the response to the pandemic, precautionary measures including social distancing have been recommended by disease and crisis management organizations including the WHO and Centers for Disease Control and Prevention (CDC). Social distancing extends to self-quarantine, avoidance of gatherings, working from home, and similar methods, which are being variably enforced by governments across the world. In the United States, most governors have ordered all non-essential businesses to close and people to shelter in place. Normal life has essentially been brought to a halt as celebrations and sporting events cease, public transit is discouraged, and individuals are advised to wear face masks and remain six feet apart.

Obvious mental health problems which have become evident due to these measures are stress, fear, and anxiety. Psychiatric disorders likely to be adversely affected by these sudden and extreme changes to daily life include overall health and anxiety disorders, social anxiety and phobic disorders, generalized anxiety disorders, somatoform disorders and abnormal illness behaviors, alcoholism and substance use disorders, eating disorders and other lifestyle related disorders.

While the current COVID-19 pandemic is a particular and rare situation, unprecedented, emergency situations will continue to occur and cause excessive levels of stress, anxiety, and depression. During these times of changing routines and mental hardship, which lead to changing sleep habits and deterioration of sleep quality, there is a need to prioritize the well-being of individuals by better managing the increased prevalence of psychological stressors caused by a deluge of alerts and media coverage regarding the crisis.

In times of such sudden and unprecedented widespread crisis, as well as other natural disasters or public health emergencies, normal routines can be upended, and stress related to the emergency can be amplified by these changes. The constant onslaught of media coverage and updates exacerbates these stresses. Depending on the dynamic nature of the situation, new information can be incoming at all times of the day, making it difficult to concentrate on things besides this news, particularly at bedtime. It is known that bedtime stress and worries are associated with increased slow wave sleep (SWS) latency, decreased sleep efficiency, and an increased wakefulness after sleep onset (WASO).

Additionally, situations that require extreme measures to mitigate safety and health concerns, such as social distancing and work from home precautions, can disrupt normal sleep architecture, resulting in longer sleep onset latency (SOL), potential fragmentation, shifting proportions of random eye movement (REM) and non-random eye movement (NREM) sleep, and shifts in bedtime and wakeup time. Indeed, studies report significant associations between poor sleep quality and work overload, work discontent, excessive demands, lack of social recognition, social isolation, and chronic worrying, a combination of which factors are likely to occur during widespread and persisting emergency situations. An insufficient buildup of sleep need can compound this abnormal sleep architecture as individuals experience reduced physical activity levels, limited light exposure, irregular eating times and patterns, and stress.

As concerns over the perceived threats associated with the emergency grow, anxiety-related behaviors, insomnia, and overall lower perceived state of health will follow. Every person may be vulnerable to the effects of widespread panic and threat. Individuals suffering from preexisting mental illness including anxiety and depression, can be affected at a larger scale especially in their sleep. Additionally, mental health related sleep disturbances such as difficulty falling asleep, persistent worrying thoughts, and nightmares can be exacerbated by the emergency situation. Frontline workers, such as health professionals in the case of public health crises, are particularly at risk of experiencing high levels of stress, anxiety, and depressive symptoms.

Accordingly, a need exists for solutions to maintain a healthy routine in order to promote the necessary recovery, which occurs during sleep. Some prior systems that fail to provide suitable solutions are:

EP2660745A2, submitted by Almosni, discloses a digital mental health behavior monitoring system and a method for determining a user's metal health by tracking a user's digital activities. Almosni does not consider specifics of the emergency situation.

US20150364057A, submitted by Catani, discloses an activity recommendation system that can positively affect a person's wellness, health, and lifestyle. Catani provides holistic lifestyle tracking and maintenance. However, Catani is not meant to accommodate mental health concerns.

US20150251074A1, submitted by Ahmed, discloses a system of recommendations for changes to sleep, recovery time, and exercise routines. Ahmed focuses on detection of and recovery from physical activity states and centers recommendations around this. Ahmed does not consider contributions from mental state.

US20110015495A1, submitted by Dothie, discloses a system for managing and improving sleep quality from objective test data on cognitive and/or psychomotor performance. Dothie provides a sleep management system. However, Dothie focuses on objective senor data rather than subjective experiences that could elevate psychological sleep factors.

### SUMMARY OF THE INVENTION

Embodiments of the present invention appropriately gate information to reduce stress and anxiety and help maintain a healthy circadian sleep timing by optimizing sleep schedules and quantifying mental fitness.

As one aspect of the present invention, a smart scheduling and information gathering system to promote sleep and mental health in unprecedented emergency situations is provided. The system may comprise a remote server, a user computing device, and at least one environmental sensor. The remote server may be communicably coupled to the user computing device and the environmental sensor. The remote server may be configured for gathering contextual event data from at least one external data source. The remote server may be configured for capturing physiological data with the environmental sensor. The remote server may be configured for generating a sleep availability rating by analyzing a sleep activity log managed by the remote server. The remote server may be configured for generating a scheduling availability rating by analyzing a current-date-and-time in relation to a user calendar managed by the remote server. The remote server may be configured for generating a behavioral recommendation and a filtering protocol by analyzing the contextual event data, the physiological data, the sleep availability rating, and the scheduling availability rating.

The system may further comprise a user computing device. The user computing device may be configured for outputting the behavioral recommendation. The user computing device may be configured for comparing incoming data to the filtering protocol in order to classify the incoming data as authorized data or unauthorized data. The user computing device may be configured for outputting the authorized data.

As another aspect of the present invention, a method for smart scheduling and information gathering to promote sleep and mental health of a user is provided. The method may comprise: providing a user account managed by at least one remote server, the user account being associated with a user computing device and at least one environmental sensor; gathering contextual event data from at least one external data source with the remote server; capturing physiological data with at least one environmental sensor; generating a sleep availability rating with the remote server by analyzing a sleep activity log; generating a scheduling availability rating with the remote server by analyzing a current-date-and-time in relation to a user calendar; generating a behavioral recommendation and a filtering protocol, with the remote server by analyzing the contextual event data, the physiological data, the sleep availability rating, and the scheduling availability rating; outputting the behavioral recommendation with the user computing device; analyzing data from the external data source through the filtering protocol with the user computing device in order to classify incoming data as authorized data or unauthorized data; and outputting the authorized data with the user computing device.

The method may further comprise prompting the user to provide user feedback. The method may further comprise receiving the user feedback and responsive thereto, updating a model.

Capturing physiological data with the at least one environmental sensor may comprise capturing physiological data with at least one wearable sensor.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are provided for the purpose of illustration and description only and are not intended as a definition of the limits of the invention.

### BRIEF DESCRIPTION OF THE DRAWINGS

FIG. 1 is a block diagram illustrating the data connections between components of an example embodiment of the present invention; and

FIG. 2 is an illustration of a curated news feed being output by a user computing device in accordance with an example embodiment of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. As used herein, “selectively coupled” means that two elements are coupled in a manner that can be uncoupled and coupled again without requiring any special tool or tools and without causing damage to either of the elements. As used herein, “permanently coupled” means that two elements are coupled in a manner that cannot be uncoupled without causing damage to one or both of the elements and/or modifying the arrangement in a manner such that the elements cannot be recoupled in the original manner without the use of further materials. For example, elements that are glued, welded, or otherwise bonded would be example of elements that are “permanently coupled”.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality) and the singular form of “a”, “an”, and “the” include plural referents unless the context clearly indicates otherwise.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Embodiments of the present invention are designed to alleviate stress and mental health issues such as those experienced by a user during an emergency. To achieve such functionality, embodiments of the present invention generally employ systems that comprise at least one remote server, a user computing device, and at least one environmental sensor. The remote server is a backend computing system used to execute data processing procedures and manage communication between the components of the present invention. In some embodiments, the remote server is at least one of the systems selected from the group comprising datacenters, cloud computing services, distributed ledgers, and application specific integrated circuit (ASIC) systems. The user computing device is an electronic device capable of receiving user input, relaying relevant information to a user, communicating with external systems, and executing the processes required to implement the present invention. In some embodiments, the user computing device includes at least one system selected from the group comprising smartphones, laptops, tablet computers, desktop computers, and wearable computers. Additionally, the user computing device is associated with a corresponding user account that is managed by the remote server. In some embodiments, the at least one environmental sensor is at least one of the sensor selected from the group comprising electrocardiography sensors, electromyography sensors, electrocochleography sensors, motion sensors, location sensors, biometric sensors, and/or temperature sensors. Additionally, the at least one environmental sensor is associated with the corresponding user account. In some embodiments the at least one environmental sensor is incorporated into a wearable device or an internet of things (IoT) device.

The remote server is configured to execute a series of processes that facilitate monitoring at least one user data point selected from the group comprising: a physiological state, a location, a virtual schedule, and at least one external content. Specifically, the remote server is configured for gathering contextual event data from at least one external data source. The contextual event data is a collection of metadata and classifiers that quantifies relevant information about the external content received from the external data source. The external content may include at least one content type selected from the group comprising images, videos, and anything otherwise available to the public. Additionally, the remote server generates the contextual event data using a natural language processing (NLP) engine. Accordingly, the remote server is able to employ a dynamic content analysis routine capable of parsing data from disparate sources.

The remote server is further configured for capturing physiological data with the at least one environmental sensor. Consequently, the present invention is able to monitor the physiological state of the user to determine an optimal time to engage in at least one activity selected from the group comprising sleeping, exercising, eating, meditating, and socializing. The remote server is further configured for generating a sleep availability rating by analyzing a sleep activity log managed by the remote server. The sleep availability rating is a quantified measure of the need for sleep at any given time. Accordingly, the remote server uses the physiological data to identify when the user is asleep or awake, and to populate the sleep activity log. Further, the remote server then correlates an over accumulation of, or excessive need for, sleep with a circadian phase of the user to determine the sleep availability rating.

The remote server is further configured for generating a scheduling availability rating by analyzing a current-date-and-time in relation to a user calendar managed by the remote server. The scheduling availability rating quantifies the amount of time the user has between the current-date-and-time and a scheduled engagement.

The present invention is designed to function as a decision assistance system that removes stress from the user's life by monitoring a plurality of personalized data points and making recommendations for activities the user can perform to improve overall health and wellness. To facilitate this, the remote server is further configured for generating a behavioral recommendation and a filtering protocol by analyzing the contextual event data, the physiological data, the sleep availability rating, and the scheduling availability rating. The behavioral recommendation presents the user with at least one activity to perform for optimized health and wellness. To achieve this functionality, the present invention uses the contextual event data, the physiological data, the sleep availability rating, and the scheduling availability rating to form a virtual model of the user. The remote server analyzes the virtual model to determine the user's mental state and physiological state. Further, the remote server analyzes the virtual model and the user's scheduling data to determine if the user has time to perform activities such as sleeping or exercising. Embodiments of the present invention use machine learning techniques to incorporate historical data into the virtual model when generating the behavioral recommendation. The user's medical data may be incorporated into the virtual model so that the behavioral recommendation is able to recommend activities that move the user toward health goals. Further, the user may be prompted, through the user computing device, to enter data relating to experiences and health goals.

The filtering protocol is informed by the behavioral recommendation and implements a content control subprocess to govern the type of content presented to the user through the user computing device. The filtering protocol employs the NLP engine to identify a set of contextual triggers to flag within the external content. The behavioral recommendation directs the filtering protocol to exclude external content that contains contextual triggers which would influence the user's emotional state in a manner contrary to a desired goal.

The user computing device serves as an interface between the user and the present invention. To facilitate such functionality, embodiments of the user computing device feature systems that include screens, speakers, wireless radios, microcontrollers and human machine interface devices. The user computing device is configured for outputting the behavioral recommendation. As a result, the user is able to be presented with images and video content in addition to audio and haptic content. The user computing device is further configured for analyzing incoming data with the filtering protocol in order to classify the incoming data as authorized data or unauthorized data. The incoming data is external content. The user computing device analyzes the incoming data using with the filtering protocol. Thereby, determining if the incoming content contains contextual triggers that would influence the user's emotional state in a manner contrary to a desired goal. These contrary pieces of content are classified as unauthorized and are filtered out of the stream of content provided to the user through the user computing device. The remaining incoming content is classified as authorized and output to the user through the computing device.

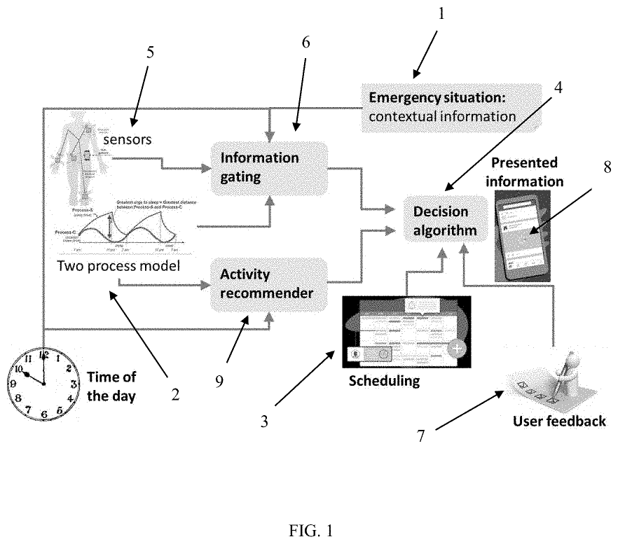

FIG. 1 shows the general interactions of the main components of a system in accordance with an exemplary embodiment of the present invention. In such example, a module 1 that contains contextual information related to the particular emergency situation is implemented as a natural processing language (NLP) system. In its simplest implementation, this is a dictionary of words with an associated frequency; for instance {‘COVID-19’: 0.5; ‘Inflammation’: 0.2; ‘ARDS’: 0.1; ‘Infection’: 0.1}.

An essential component of the present invention is the module 2 (“Two process model”) which utilizes a sleep/wake regulation model that considers both circadian and sleep-homeostasis factors to predict the need for sleep at a given time. For instance, if the user has been awake for more than 24 hours, the model can quantify the accumulated excessive need for sleep while also taking into account the circadian phase of the user. For instance, due to 24-hour rhythms in sleep-promoting neuro-transmitters and hormones, it is easier to fall asleep in the evening compared to the early morning.

The schedule 3 of the user (e.g. in the form of an electronic calendar) as well as the current time are also taken into account to organize the information/recommendations presented to the user by a decision algorithm.

A decision algorithm 4 aggregates the information from multiple sources and evaluates the opportunity, necessity, and ability to sleep. In evaluating the opportunity to sleep, the decision algorithm 4 considers the user schedule and estimates the shortest and longest possible duration of sleep. The necessity to sleep is estimated by the decision algorithm 4 from the Two-process model 2 and its simplest implementation considers how long the user has been awake. The ability to sleep aspect is evaluated by the decision algorithm 4 using physiological sensors 5, e.g. heart-rate and heart-rate variability (if available). The information 8 presented to the user is tailored such that it does not impair the ability of the user to sleep. For instance, it may not be critical for the user to know about the latest news regarding the emergency situation at the time of desired sleep. Alternatively, that information could be presented to them at the time of wake-up. A contextual-information module 6 provides a dictionary-like structure that enables the decision algorithm 4 to automatically quantify the extent to which a given content (video, audio, or text) is related to the emergency situation.

In some embodiments, the system incorporates user feedback 7 to enhance the decision algorithm 4. The system may prompt for such feedback 7 from the user at specific times. In the event that the system would need more information, there would be a prompt for the user to confirm any particular finding. For example, if the user's heart rate is elevated, the system would confirm with the user if they are involved in any rigorous physical activity. This would help in increasing the specificity of the results of the decision algorithm 4. As the decision algorithm 4 gathers more data over time, the system would have the capability to run a prediction algorithm based on the available inputs.

In example embodiments, the solution is intended to be used in the context of a pandemic situation like COVID-19, MERS, SARS, etc., by individuals who may or may not be clinically diagnosed with a mental health disorder such as anxiety, clinical depression, bipolar disorder, obsessive-compulsive disorder, post-traumatic stress disorder, etc. Sleep problems are particularly common in individuals with a mental health disorder. Disturbed sleep quality, difficulty falling asleep and/or maintaining sleep, nightmares, vivid dreams, etc. are all consequences of the mental battles that occur in these individuals, amplified during a pandemic situation. In this scenario, the system helps users with their sleep schedules. More specifically, embodiments of the present invention help to optimize a user's sleep time(s) by recommending appropriate activities 9 depending on the time of the day and the user's circadian phase. The decision algorithm 4 ensures that the “smart scheduler” recommends activities 12 like exercise, mindfulness (paced breathing, progressive muscle relaxation, yoga), reading, timing of meals. Such recommendations are based on a plurality of different inputs, including but not limited to time of the day, circadian/two process models, the user's personal schedules, and physiological data (heart rate, respiratory rate, heart rate variability, oxygen saturation, temperature, etc.) acquired from wearable and standalone devices (e.g., physiological sensors 5). By recommending these timely activities, the system ensures that there is a significant buildup of sleep pressure by the time the user is scheduled to go to bed.

An essential part of the proposed system is the filtering and prioritizing of information that is readily available to the users (news apps, social media, etc.), so that the information the user receives does not cause any unnecessary panic. This helps reduce a significant domino effect that mental stress can have on sleep. The decision algorithm prioritizes information from various applications and ensures that the most important, relevant and necessary information appears first. For instance, during a pandemic if the user's zip code is marked as a hotspot for infection, this information needs to reach the user before it's too late. This will be prioritized over other pieces of information such as overall number of positive cases or deaths.

Wearable sensor data, when available, is synced to be able to perform the filtering of data and scheduling appropriately. Signals like heart rate, respiratory rate, etc. can be vital in making decisions relating to prioritizing information and in suggesting appropriate activities. For example, if the user's heart rate is elevated, the system would suggest the user to do some kind of relaxation activity, i.e., breathing, meditation or aroma therapy to help control the anxious feeling; meanwhile also avoiding notifications such as news flash so as to alleviate and not exacerbate the condition.

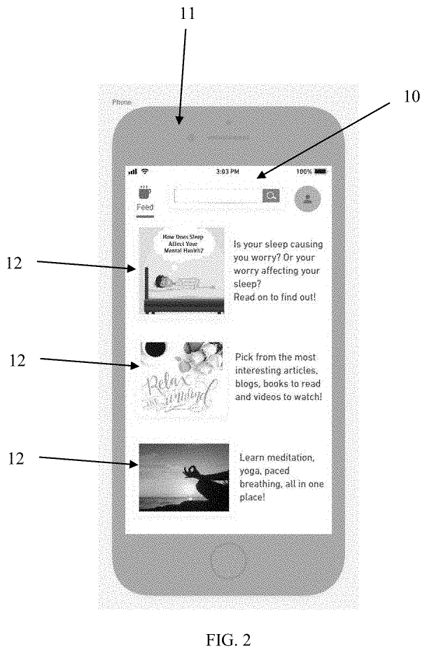

Although a preferred embodiment of the present invention uses a smartphone phone app 10 as a medium (e.g., see FIG. 2), other platforms can also be utilized to communicate or notify the users. In scenarios where smartphones 11 are not available, computer software or even telehealth could be used as a medium to communicate with the user, help in scheduling activities, and process any information received. In any case, the system would have the capability to customize the recommendations and scheduling to everyone based on their baseline data.

From the foregoing it is thus to be appreciated that embodiments of the present invention provide systems and methods that improve a user's sleep and mental health.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A smart scheduling and information gathering system to promote sleep and mental health in unprecedented emergency situations, the system comprising:
at least one remote server;
a user computing device; and
at least one environmental sensor,
wherein the remote server is communicably coupled to the user computing device and the at least one environmental sensor,
wherein the remote server is configured for:
gathering contextual event data from at least one external data source;
capturing physiological data with the at least one environmental sensor;
generating a sleep availability rating by analyzing a sleep activity log managed by the remote server;
generating a scheduling availability rating by analyzing a current-date-and-time in relation to a user calendar managed by the remote server; and
generating a behavioral recommendation and a filtering protocol by analyzing the contextual event data, the physiological data, the sleep availability rating, and the scheduling availability rating, and
wherein the user computing device is configured for:
outputting the behavioral recommendation;
analyzing incoming data with the filtering protocol in order to classify the incoming data as authorized data or unauthorized data; and
outputting the authorized data.

2. The system of claim 1, wherein the user computing device prompts to enter at least one user feedback.

3. The system of claim 1, wherein the at least one environmental sensor is at least one wearable sensor.

4. The system of claim 1, wherein the user computing device implements the filtering protocol through a NLP engine.

5. A method for smart scheduling and information gathering to promote sleep and mental health of a user, the method comprising:
providing a user account managed by at least one remote server, the user account being associated with a user computing device and at least one environmental sensor;
gathering contextual event data from at least one external data source with the remote server;
capturing physiological data with at least one environmental sensor;
generating a sleep availability rating with the remote server by analyzing a sleep activity log;
generating a scheduling availability rating with the remote server by analyzing a current-date-and-time in relation to a user calendar;
generating a behavioral recommendation and a filtering protocol, with the remote server by analyzing the contextual event data, the physiological data, the sleep availability rating, and the scheduling availability rating;
outputting the behavioral recommendation with the user computing device;
analyzing data from the external data source through the filtering protocol with the user computing device in order to classify incoming data as authorized data or unauthorized data; and
outputting the authorized data with the user computing device.

6. The method of claim 5, further comprising prompting the user to provide user feedback.

7. The method of claim 6, further comprising receiving the user feedback and responsive thereto, updating a model.

8. The method of claim 5, wherein capturing physiological data with at least one environmental sensor comprises capturing physiological data with at least one wearable sensor.
